(19) United States
(12) Patent Application Publication
(10) Pub. No.: US 2024/0148637 A1
(43) Pub. Date: May 9, 2024

(54) MINERAL OIL-FREE COSMETIC W/O EMULSION

(71) Applicant: BEIERSDORF AG, Hamburg (DE)

(72) Inventors: Sebastian TIMM, Hamburg (DE); Perta KOCH, Hamburg (DE); Yang XU, Hamburg (DE)

(21) Appl. No.: 18/272,634

(22) PCT Filed: Jan. 13, 2022
(86) PCT No.: PCT/EP2022/050606

§ 371 Date: Jul. 17, 2023

(30) Foreign Application Priority Data
Jan. 20, 2021 (DE) 10 2021 000 268.7

(51) Int. Cl. A61K 8/92; A61K 8/06; A61K 8/19; A61K 8/34; A61K 8/365; A61K 8/37; A61K 8/42; A61K 8/55; A61K 8/67; A61Q 19/00

(52) U.S. Cl. CPC A61K 8/922; A61K 8/064; A61K 8/19; A61K 8/342; A61K 8/365; A61K 8/37; A61K 8/375; A61K 8/42; A61K 8/553; A61K 8/676; A61Q 19/00; A61K 2800/10; A61K 2800/30

(57) ABSTRACT

cosmetic water-in-oil emulsion (W/O emulsion) containing triglycerides, the preparation being free from mineral oil, paraffin wax, microcrystalline wax, shellac wax and polyethylene waxes, polyacrylates, crosslinked acrylate/C10-C30 alkylacrylate polymers, vinylpyrrolidone/hexadecene copolymers, 3-(4-methylbenzylidene)-campher, 2-hydroxy-4-methoxybenzophenone (INCI: oxybenzone), 2-ethylhexyl 4-methoxycinnamate (INCI: octylmethoxycinnamate), ethylhexyl-2-cyano-3,3-diphenylacrylate (INCI: octocrylene), parabens (in particular methyl-, propyl- and butylparaben), methylisothiazolinone, chloromethylisothiazolinone and DMDM-hydantoin, polyethylene glycol ethers or polyethylene glycol esters.

The present invention relates to a cosmetic water-in-oil emulsion (W/O emulsion) comprising triglycerides, the preparation being free of mineral oil, paraffin wax, microcrystalline wax, shellac wax and polyethylene waxes, polyacrylates, crosslinked acrylate/C10-C30 alkyl acrylate polymers, vinylpyrrolidone/hexadecene copolymers, 3-(4-methylbenzylidene)camphor, 2-hydroxy-4-methoxybenzophenone (INCI: Oxybenzone), 2-ethylhexyl 4-methoxycinnamate (INCI: Octylmethoxycinnamate), ethylhexyl 2-cyano-3,3-diphenylacrylate (INCI: Octocrylene), parabens (especially ethyl-, propyl-, and butylparaben), methylisothiazolinone, chloromethylisothiazolinone and DMDM hydantoin, polyethylene glycol ethers or polyethylene glycol esters.

The desire to look beautiful and attractive is by nature ingrained in humans. Although ideals of beauty have changed over time, the pursuit of an immaculate appearance has always been the goal in humans. An essential part of a beautiful and attractive appearance is the condition and complexion of the skin.

In order for the skin to be able to perform the full range of its biological functions, it requires regular cleansing and care. Skincare products, usually creams, ointments or lotions, mostly serve for moisturizing and refatting of the skin. Active substances are commonly added thereto, which are intended to regenerate the skin and for example to prevent and reduce its premature aging (for example the development of fine lines and wrinkles).

In addition to the cleansing and care of the skin, cosmetics have an aesthetic role too. They are intended to “improve” the external appearance of the user in accordance with the prevailing cultural perceptions. Cosmetics thus perform a psychological/social function, since they increase the (visual) attractiveness of users.

Recent times have seen a growing trend toward “natural” cosmetics, the ingredients of which should no longer come from petroleum products or be chemically synthesized. The search for alternative ingredients that meet these criteria poses particular challenges for product developers. This is because replacing known ingredients such as mineral oils, silicone oils, and polyacrylates almost invariably comes at the cost of disadvantages in respect of product use. The preparations become unattractive from a sensory viewpoint, which becomes disagreeably noticeable for example when the preparation is spread on the skin and through an inadequate absorption capacity.

It was therefore an object of the present invention to develop a cosmetic emulsion (in particular a W/O emulsion) that is free of mineral oils, silicone oils, and polyacrylates and that has the same sensory properties as conventional cosmetics that contain these ingredients. These preparations should ideally be “vegan”, i.e., contain no ingredients of animal origin. A further aim was the development of a preparation, the ingredients of which are of vegetable origin, where vegetable origin is to be understood as encompassing also substances the starting material of which comes from plants and is then subjected to one or more derivatization steps (for example hydrolysis, esterification).

Cosmetic water-in-oil emulsions (W/O emulsions) are used in particular as a base for preparations serving for moisturizing and refatting of the skin. Besides its role as a lipid donor, the emulsion base also serves as a vehicle for skin moisturizing agents such as glycerol and panthenol (also called dexpanthenol) that are present in the water phase of the emulsion. In order to become cosmetically/dermatologically active, these substances must then be released from the water phase by evaporation of the water vehicle. The rate of evaporation of the water is therefore usually the rate-determining step for the onset of action of the active ingredients present in the water phase.

It was therefore an object of the present invention to develop a water-in-oil emulsion (W/O emulsion) in which the active substances present in the water phase (in particular glycerol and panthenol) are released more quickly.

In particular, it was an object of the present invention to increase the rate of evaporation of the water from the emulsion.

The objects are surprisingly achieved by a cosmetic water-in-oil emulsion (W/O emulsion) comprising triglycerides, the preparation being free of mineral oil, paraffin wax, microcrystalline wax, shellac wax and polyethylene waxes, polyacrylates, crosslinked acrylate/C10-C30 alkyl acrylate polymers, vinylpyrrolidone/hexadecene copolymers, 3-(4-methylbenzylidene)camphor, 2-hydroxy-4-methoxybenzophenone (INCI: Oxybenzone), 2-ethylhexyl 4-methoxycinnamate (INCI: Octylmethoxycinnamate), ethylhexyl 2-cyano-3,3-diphenylacrylate (INCI: Octocrylene), parabens (especially ethyl-, propyl-, and butylparaben), methylisothiazolinone, chloromethylisothiazolinone and DMDM hydantoin, polyethylene glycol ethers or polyethylene glycol esters.

The achievement of the object was accordingly not obvious to those skilled in the art, in particular because this would have necessitated assuming that water egress and thus the rate of evaporation is more strongly hindered by the formation of hydrogen bonds to the oxygen atoms of the triglyceride esters than occurs in the mineral oils, paraffin waxes, and microcrystalline waxes in conventional W/O emulsions. However, this is not the case.

It is advantageous according to the invention when the amount of the water phase is 35% to 75% by weight and the amount of the oil phase 25% to 65% by weight, based on the total amount of the preparation.

It is preferable according to the invention when the preparation is free of aliphatic and aromatic hydrocarbons.

Embodiments of the present invention that are advantageous according to the invention are characterized in that the preparation additionally contains straight-chain and/or branched fatty alcohols.

It is preferable according to the invention when octyldodecanol, cetyl alcohol and/or stearyl alcohol are employed as straight-chain and/or branched fatty alcohols. It is also preferable according to the invention when these straight-chain and/or branched fatty alcohols are used in a total concentration of 0.3% to 2.5% by weight based on the total weight of the preparation.

Embodiments of the present invention that are advantageous according to the invention are characterized in that preparation is emulsified with the emulsifier diisostearoyl polyglyceryl-3 dimer dilinoleate. This can be purchased for example under the trade name Isolan PDI from Evonik. It is preferable according to the invention when the preparation contains diisostearoyl polyglyceryl-3 dimer dilinoleate in a concentration of 1.5% to 2.5% by weight based on the total weight of the preparation.

It is advantageous according to the invention when the content of triglycerides in the emulsion is from 10% to 30% by weight based on the total weight of the preparation. According to the invention, a concentration range of from 20% to 27% by weight based on the total weight of the preparation is preferable here.

Advantageous embodiments of the present invention are further characterized in that preparation contains lecithin and/or glycerol. The preferred use concentration for lecithin is according to the invention 0.0005% to 0.1% by weight based on the total weight of the preparation.

The preferred use concentration for glycerol is according to the invention 2% to 6% by weight based on the total weight of the preparation.

In addition, it is advantageous according to the invention when the preparation according to the invention contains one or more esters selected from the group of the compounds ascorbyl palmitate, tocopheryl acetate, and cetyl palmitate.

The preferred use concentration for ascorbyl palmitate is according to the invention 0.0003% to 0.1% by weight based on the total weight of the preparation.

The preferred use concentration for tocopheryl acetate is according to the invention 0.0001% to 0.1% by weight based on the total weight of the preparation.

The preferred use concentration for cetyl palmitate is according to the invention 0.005% to 1.0% by weight based on the total weight of the preparation.

It is advantageous according to the invention when preparation contains one or more salts selected from the group of the compounds magnesium sulfate, sodium anisate, and sodium citrate. Sodium citrate, which can also be used in the form of its free acid (citric acid), can be used here, which is preferable according to the invention.

The preferred use concentration for magnesium sulfate is according to the invention 0.3% to 1.5% by weight based on the total weight of the preparation.

The preferred use concentration for sodium anisate is according to the invention 0.002% to 0.9% by weight based on the total weight of the preparation.

The preferred use concentration for citric acid or sodium citrate is according to the invention 0.001% to 0.8% by weight based on the total weight of the preparation.

Advantageous embodiments of the present invention are characterized in that preparation contains phenoxyethanol and/or panthenol.

The preferred use concentration for phenoxyethanol is according to the invention 0.01% to 0.7% by weight based on the total weight of the preparation.

The preferred use concentration for panthenol is according to the invention 0.05% to 0.94% by weight based on the total weight of the preparation.

According to the invention, the use of a combination of panthenol, more particularly D-panthenol, and glycerol is particularly preferred.

The embodiments of the present invention that are advantageous according to the invention are characterized in that the triglycerides are selected from mixtures of hydrogenated rapeseed oil, rapeseed oil, caprylic/capric acid triglycerides, sunflower oil, hydrogenated coconut glycerides, hydrogenated castor oil, and shea butter, the mixture preferably consisting of a plurality or all of these triglyceride sources.

When the preparation of the invention contains hydrogenated rapeseed oil (INCI: Hydrogenated Rapeseed Oil), this is according to the invention preferably used in a concentration of 4% to 15% by weight based on the total weight of the preparation.

When the preparation of the invention contains caprylic/capric acid triglycerides (INCI: Caprylic/Capric Triglycerides), this is according to the invention preferably used in a concentration of 5.1% to 10% by weight based on the total weight of the preparation.

When the preparation of the invention contains rapeseed oil (INCI: Brassica Campestris Seed Oil), this is according to the invention preferably used in a concentration of 2% to 4% by weight based on the total weight of the preparation.

When the preparation of the invention contains sunflower oil (more particularly oil having the INCI Helianthus Annuus Hybrid Oil), this is according to the invention preferably used in a concentration of 1.6% to 5% by weight based on the total weight of the preparation.

The W/O emulsion of the invention is usually present in the form of a cream.

### Comparative Experiment

The following formulations were prepared and the rate of water loss determined gravimetrically.

| | Cream OP 1483 | Cream OP 1702 |
|---|---|---|
| Ingredients (INCI) | (% by wt.) | (% by wt.) |
| Aqua | ad 100 | ad 100 |
| Hydrogenated Rapeseed Oil, | | 13.5 |
| Brassica Campestris Seed Oil, | | |
| Hydrogenated Coco Glycerides, | | |
| Butyrospermum Parkii Oil | | |
| Paraffinum Liquidum, Cera | 13.5 | |
| Microcristallina, Tocopheryl | | |
| Acetate, BHT | | |
| Diisostearoyl Polyglyceryl-3 | 2 | 2 |
| Dimer Dilinoleate | | |
| Caprylic/Capric Triglycerides, | 10.76 | 10.76 |
| Helianthus Annuus Hybrid Oil, | | |
| Hydrogenated Castor Oil* | | |
| Cetearyl Alcohol, Octyldodecanol* | 0.92 | 0.92 |
| Cetyl Palmitate | 0.3 | 0.3 |
| Glycerin | 4 | 4 |
| Panthenol | 0.1 | 0.1 |
| Magnesium Sulfate | 0.7 | 0.7 |
| Lecithin, Ascorbyl Palmitate, | 0.43 | 0.43 |
| Phenoxyethanol, Pantolactone, | | |
| Sodium Anisate, Citric Acid, | | |
| Parfum* | | |

*= the identical mixture was employed in both recipes.

Procedure:

For each emulsion, the backs of 10 petri dishes were each covered with cream. This simulated rubbing the lotion into the skin (circular movements). Before application, the empty weight of each petri dish was determined. The weight was then determined after the application of the cream. The weight of cream applied is calculated as the difference between the two measured values.

TABLE 1
Determination of the weight of cream containing mineral oil

| Cream OP 1483 | Weight (g) | Weight + cream (g) | Cream (g) |
|---|---|---|---|
| 1 | 46.74 | 47.32 | 0.58 |
| 2 | 46.06 | 46.56 | 0.5 |
| 3 | 46.1 | 46.75 | 0.65 |
| 4 | 46.08 | 46.72 | 0.64 |
| 5 | 46.4 | 47.06 | 0.66 |
| 6 | 46.35 | 46.96 | 0.61 |
| 7 | 46.09 | 46.79 | 0.7 |
| 8 | 46.46 | 47.23 | 0.77 |
| 9 | 46.23 | 47.04 | 0.81 |
| 10 | 46.79 | 47.49 | 0.7 |

TABLE 2
Determination of the weight of mineral oil-free cream

| Cream OP 1702 | Weight (g) | Weight + cream (g) | Cream (g) |
|---|---|---|---|
| 1 | 46 | 46.53 | 0.53 |
| 2 | 46.02 | 46.78 | 0.76 |
| 3 | 46.71 | 47.19 | 0.48 |
| 4 | 45.85 | 46.31 | 0.46 |
| 5 | 45.84 | 46.44 | 0.6 |
| 6 | 46.03 | 46.55 | 0.52 |
| 7 | 46.55 | 47.25 | 0.7 |
| 8 | 45.97 | 46.72 | 0.75 |
| 9 | 46.1 | 46.73 | 0.63 |
| 10 | 46.56 | 47.27 | 0.71 |

After the Petri dishes had been covered with the respective cream, the weight was determined at the following times:
30 minutes
60 minutes
90 minutes
180 minutes
300 minutes

The tables below show the respective weights of the creams at the measurement times listed above.

TABLE 3
Weight of the samples containing mineral
oil at the different measurement times

| Cream | Cream | Cream | Cream | Cream | Cream | Cream |
|---|---|---|---|---|---|---|
| OP | (g) | (g) | (g) | (g) | (g) | (g) |
| 1483 | 0 min | 30 min | 60 min | 90 min | 180 min | 300 min |
| 1 | 0.58 | 0.56 | 0.55 | 0.55 | 0.52 | 0.49 |
| 2 | 0.5 | 0.48 | 0.47 | 0.47 | 0.45 | 0.42 |
| 3 | 0.65 | 0.63 | 0.64 | 0.63 | 0.6 | 0.58 |
| 4 | 0.64 | 0.63 | 0.62 | 0.61 | 0.59 | 0.56 |
| 5 | 0.66 | 0.65 | 0.64 | 0.64 | 0.62 | 0.59 |
| 6 | 0.61 | 0.59 | 0.58 | 0.59 | 0.57 | 0.54 |
| 7 | 0.7 | 0.68 | 0.67 | 0.68 | 0.68 | 0.64 |
| 8 | 0.77 | 0.75 | 0.76 | 0.75 | 0.74 | 0.71 |
| 9 | 0.81 | 0.79 | 0.78 | 0.78 | 0.76 | 0.73 |
| 10 | 0.7 | 0.76 | 0.67 | 0.66 | 0.65 | 0.62 |

TABLE 4
Weight of the mineral oil-free samples
at the different measurement times

| Cream | Cream | Cream | Cream | Cream | Cream | Cream |
|---|---|---|---|---|---|---|
| OP | (g) | (g) | (g) | (g) | (g) | (g) |
| 1702 | 0 min | 30 min | 60 min | 90 min | 180 min | 300 min |
| 1 | 0.53 | 0.43 | 0.37 | 0.35 | 0.27 | 0.21 |
| 2 | 0.76 | 0.61 | 0.58 | 0.54 | 0.46 | 0.37 |
| 3 | 0.48 | 0.35 | 0.3 | 0.27 | 0.21 | 0.18 |
| 4 | 0.46 | 0.32 | 0.3 | 0.27 | 0.21 | 0.17 |
| 5 | 0.6 | 0.46 | 0.41 | 0.38 | 0.3 | 0.24 |
| 6 | 0.52 | 0.39 | 0.34 | 0.3 | 0.23 | 0.18 |
| 7 | 0.7 | 0.55 | 0.51 | 0.47 | 0.39 | 0.31 |
| 8 | 0.75 | 0.59 | 0.55 | 0.52 | 0.44 | 0.34 |
| 9 | 0.63 | 0.5 | 0.44 | 0.42 | 0.33 | 0.25 |
| 10 | 0.71 | 0.58 | 0.52 | 0.49 | 0.4 | 0.31 |

As can be seen from Table 3, the loss in weight of the samples containing mineral oil is only very small. This is not the case with the mineral oil-free cream (Table 4). A marked loss in weight, i.e., loss of water, is already evident within the first 30 minutes. Tables 5 and 6 below show the percentage loss in weight in each case.

TABLE 5
Loss in weight in % of the mineral oil-containing creams

| | Loss in | Loss in | Loss in | Loss in | Loss in |
|---|---|---|---|---|---|
| Cream | weight | weight | weight | weight | weight |
| OP | (%) | (%) | (%) | (%) | (g) |
| 1483 | 30 min | 60 min | 90 min | 180 min | 300 min |
| 1 | 3 | 5 | 5 | 10 | 16 |
| 2 | 4 | 6 | 6 | 10 | 16 |
| 3 | 3 | 2 | 3 | 8 | 11 |
| 4 | 2 | 3 | 5 | 8 | 12 |
| 5 | 2 | 3 | 3 | 6 | 11 |
| 6 | 3 | 5 | 3 | 7 | 11 |
| 7 | 3 | 4 | 3 | 3 | 9 |
| 8 | 3 | 1 | 3 | 4 | 8 |
| 9 | 2 | 4 | 4 | 6 | 10 |
| 10 | 3 | 4 | 4 | 7 | 11 |

After 30 minutes the loss in weight of the samples was on average only 3%. After 90 minutes it is just 4%.

TABLE 6
Loss in weight in % of the mineral oil-free cream

| Cream OP | Cream (g) | Cream (g) | Cream (g) | Cream (g) | Cream (g) |
|---|---|---|---|---|---|
| 1483 | 30 min | 60 min | 90 min | 180 min | 300 min |
| 1 | 19 | 30 | 34 | 49 | 60 |
| 2 | 20 | 24 | 29 | 39 | 51 |
| 3 | 27 | 38 | 44 | 56 | 62 |
| 4 | 30 | 35 | 41 | 54 | 63 |
| 5 | 23 | 32 | 37 | 50 | 60 |
| 6 | 25 | 35 | 42 | 56 | 65 |
| 7 | 21 | 27 | 33 | 44 | 56 |
| 8 | 21 | 27 | 31 | 41 | 55 |
| 9 | 21 | 30 | 33 | 48 | 60 |
| 10 | 18 | 27 | 31 | 44 | 56 |
| Mean | 23 | 30 | 35 | 84 | 59 |

After 30 minutes the loss in weight, i.e., loss of water, of the mineral oil-free samples was already 23%.

### EXAMPLES

The examples below are intended to elucidate the present invention without limiting it. Unless otherwise stated, all amounts, proportions, and percentages are based on the weight and total amount and on the total weight of the preparations.

| Ingredients (INCI) | Example 1 | Example 2 | Example 3 | Example 4 |
|---|---|---|---|---|
| Water | ad 100 | ad 100 | ad 100 | ad 100 |
| Hydrogenated Rapeseed Oil | 8.47 | 6.12 | 4.15 | 7.10 |
| Brassica Campestris Seed Oil | 2.55 | 2.28 | 3.22 | 3.32 |
| Hydrogenated Coco Glycerides | 1.64 | 1.53 | 1.85 | 1.27 |
| Butyrospermum Parkii Oil | 1.43 | 1.94 | 1.75 | 1.79 |
| Diisostearoyl Polyglyceryl-3 Dimer | 1.60 | 1.80 | 2.20 | 2.00 |
| Dilinoleate | | | | |
| Caprylic/Capric Triglycerides | 5.50 | 9.50 | 7.00 | 5.70 |
| Helianthus Annuus Hybrid Oil | 4.00 | 2.00 | 4.80 | 4.75 |
| Hydrogenated Castor Oil | 0.40 | 0.40 | 0.40 | 0.40 |
| Cetearyl Alcohol | 0.33 | 0.70 | 0.50 | 0.30 |
| Octyldodecanol | 0.66 | 0.80 | 1.00 | 0.75 |
| Cetyl Palmitate | 0.90 | 0.50 | 0.70 | 0.10 |
| Lecithin | 0.0055 | 0.0073 | 0.0033 | 0.0068 |
| Ascorbyl Palmitate | 0.0045 | 0.0033 | 0.0077 | 0.0041 |
| Parfum | 0.17 | 0.20 | 0.22 | 0.15 |
| Glycerin | 3.00 | 4.50 | 5.50 | 2.50 |
| Panthenol | 0.80 | 0.50 | 0.40 | 0.20 |
| Magnesium Sulfate | 0.50 | 0.60 | 0.80 | 0.70 |
| Phenoxyethanol | 0.20 | 0.15 | 0.10 | 0.02 |
| Pantolactone | 0.0213 | 0.013 | 0.0106 | 0.005 |
| Sodium Anisate | 0.10 | 0.15 | 0.20 | 0.06 |
| Citric Acid | 0.04 | 0.06 | 0.08 | 0.02 |

1.-12. (canceled)

13. A cosmetic water-in-oil (W/O) emulsion, wherein the emulsion comprises one or more triglycerides and is free of mineral oil, paraffin wax, microcrystalline wax, shellac wax, polyethylene waxes, polyacrylates, crosslinked acrylate/C10-C30 alkyl acrylate polymers, vinylpyrrolidone/hexadecene copolymers, 3-(4-methylbenz ylidene)camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethyl-isothiazolinone, DMDM hydantoin, polyethylene glycol ethers and polyethylene glycol esters.

14. The emulsion of claim 13, wherein the emulsion is also free of aliphatic and aromatic hydrocarbons.

15. The emulsion of claim 13, wherein the emulsion additionally comprises one or more straight-chain and/or branched fatty alcohols.

16. The emulsion of claim 15, wherein the straight-chain and/or branched fatty alcohols comprise one or more of octyldodecanol, cetyl alcohol or stearyl alcohol.

17. The emulsion of claim 15, wherein the emulsion comprises from 0.3% to 2.5% by weight of the one or more straight-chain and/or branched fatty alcohols, based on a total weight of the emulsion.

18. The emulsion of claim 13, wherein the emulsion is emulsified with diisostearoyl polyglyceryl-3 dimer dilinoleate.

19. The emulsion of claim 18, wherein the emulsion comprises from 1.5% by weight to 2.5% by weight of diisostearoyl polyglyceryl-3 dimer dilinoleate, based on a total weight of the preparation.

20. The emulsion of claim 13, wherein the emulsion comprises from 10% by weight to 30% by weight of triglycerides, based on a total weight of the preparation.

21. The emulsion of claim 20, wherein the emulsion comprises from 20% by weight to 27% by weight of triglycerides.

22. The emulsion of claim 13, wherein the emulsion comprises lecithin and/or glycerol.

23. The emulsion of claim 22, wherein the emulsion comprises from 0.0005% to 0.1% by weight of lecithin, based on at total weight of the emulsion.

24. The emulsion of claim 22, wherein the emulsion comprises from 2% to 6% by weight of glycerol, based on a total weight of the emulsion.

25. The emulsion of claim 13, wherein the emulsion comprises from 35% to 75% by weight of aqueous phase and from 25% to 65% by weight of oil phase, based on a total weight of the emulsion.

26. The emulsion of claim 13, wherein the emulsion further comprises one or more of ascorbyl palmitate, tocopheryl acetate, cetyl palmitate.

27. The emulsion of claim 13, wherein the emulsion further comprises one or more of magnesium sulfate, sodium anisate, sodium citrate.

28. The emulsion of claim 13, wherein the emulsion further comprises phenoxyethanol and/or panthenol.

29. The emulsion of claim 13, wherein the triglycerides comprise two or more of hydrogenated rapeseed oil, rapeseed oil, caprylic/capric acid triglycerides, sunflower oil, hydrogenated coco glycerides, hydrogenated castor oil, shea butter.

30. The emulsion of claim 29, wherein the triglycerides comprise hydrogenated rapeseed oil, rapeseed oil, caprylic/capric acid triglycerides, sunflower oil, hydrogenated coco glycerides, hydrogenated castor oil, shea butter.

31. The emulsion of claim 29, wherein the emulsion further comprises two or more of cetearyl alcohol, octyldodecanol, cetyl palmitate, lecithin, ascorbyl palmitate, glycerin, panthenol, magnesium sulfate, phenoxyethanol, pantenol, sodium anisate, citric acid.

32. The emulsion of claim 13, wherein the emulsion is present in the form of a cream.
